(19) United States
(12) Patent Application Publication
(10) Pub. No.: US 2010/0292532 A1
(43) Pub. Date: Nov. 18, 2010

(54) Endoscopic Vessel Dissector With Side Entry

(75) Inventors: Randal J. Kadykowski, South Lyon, MI (US); Hideyuki Kasahara, Tokyo (JP); Takahiro Kogasaka, Tokyo (JP)

Correspondence Address: TERUMO CARDIOVASCULAR SYSTEMS CORPORATION;ATTN: GAEL DIANE TISACK, 6200 JACKSON ROAD, ANN ARBOR, MI, 48103, US

(73) Assignees: TERUMO CARDIOVASCULAR SYSTEMS CORPORATION, Ann Arbor, MI (US); OLYMPUS MEDICAL SYSTEMS CORPORATION, SHIBUYA-KUTOKYO (JP)

(21) Appl. No.: 12/465,784

(22) Filed: May 14, 2009

(51) Int. Cl. A61B 1/012; A61B 1/32

(52) U.S. Cl. 600/104; 600/210

(57) ABSTRACT

A blunt dissector for endoscopic vein harvesting has a generally-cylindrical sheath for retaining an elongated endoscope. The sheath is elongated between a distal end and a proximal end. A transparent tip is disposed at the distal end of the sheath. A handle is disposed at the proximal end of the sheath. The sheath includes a selectably closable longitudinal slot having an opened configuration such that the endoscope can be inserted radially into the sheath. The longitudinal slot has a closed configuration that retains the endoscope within the sheath and presents a substantially smooth outer surface for use during dissection.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to endoscopic vein harvesting using a blunt dissector, and, more specifically, to a dissector instrument capable of receiving an endoscope via side entry.

Harvested vessels from medical patients are used in many surgical procedures, including use as a coronary artery bypass graft, or in other cardiovascular procedures. In vascular and cardiovascular procedures, a blood vessel or vessel section, such as an artery or vein, is “harvested” (i.e., removed) from its natural location in a patient's body and is used elsewhere in the body. For example, in coronary artery bypass grafting surgery, the harvested blood vessel is used to form a bypass between an arterial blood source and one or more coronary arteries. Among the preferred sources for the vessels to be used as the bypass graft are the saphenous vein in the leg and the radial artery in the arm.

Endoscopic surgical procedures for harvesting a section of a blood vessel (e.g., the saphenous vein) subcutaneously have been developed in order to avoid disadvantages and potential complications of full surgical openings to reach the blood vessel. In the past, the harvesting was done through a continuous incision (e.g., along the leg) that exposed the full length of the desired vein section. The continuous incision had been necessary in order to provide adequate exposure for visualizing the vein and for introducing the surgical instruments to seal and sever the tissue and side branches of the vessel. A more preferred, minimally-invasive technique employs a small incision for locating the desired vessel and for introducing one or more endoscopic devices into the small incision. For example, commercially available products for performing the endoscopic blood vessel harvesting procedure include a number of separate endoscopic devices that are each inserted into the patient. These endoscopic products include, for example, an insufflation mechanism having plastic tubing to supply air or CO₂ to insufflate the subcutaneous area; an endoscope having a camera and light cables in order to visualize both the dissection and harvesting procedures; a dissector mechanism to dissect or separate the vessel from surrounding tissues in the body; and a harvester mechanism to seal and sever any branches from the vessel and to remove the vessel from the body.

One particular example of an endoscopic system is the VirtuoSaph™ Endoscopic Vein Harvesting System available from Terumo Cardiovascular Systems Corporation of Ann Arbor, Mich. This system includes a dissector rod having an atraumatic tip at the distal end. The tip is transparent so that the areas being dissected can be visualized using a rigid endoscope mounted in the hollow interior of the dissector rod. A handle at the proximal end of the dissector rod accommodates the eyepiece and light source connector of the endoscope. The endoscope is reusable for many surgeries, but the dissector rod is typically disposed of after a single use. In preparation for dissection, the endoscope is inserted axially into a receiving channel at the proximal end of the dissector rod and continuing the insertion until the head portion of the endoscope snaps into a retained position within the handle. Thus, the endoscope and dissector rod must be brought end to end and held in a coaxial manner in order to assemble them. This inefficient use of space may be undesirable in a cramped operating room, and the movements required for doing the assembly can be awkward or over-strenuous in some situations. In addition, assembly difficulties or errors can result errors can result from the inability to see the interaction of the endoscope with the channel as it is inserted.

### SUMMARY OF THE INVENTION

The present invention has the advantage of providing full visualization of the assembly operation of an endoscope into a dissector, and the advantage of a compact assembly operation with improved ergonomics.

In one aspect of the invention, a blunt dissector for endoscopic vein harvesting comprises a generally-cylindrical sheath for retaining an elongated endoscope. The sheath is elongated between a distal end and a proximal end. A transparent tip is disposed at the distal end of the sheath. A handle is disposed at the proximal end of the sheath. The sheath includes a selectably closable longitudinal slot having an opened configuration such that the endoscope can be inserted radially into the sheath. The longitudinal slot has a closed configuration that retains the endoscope within the sheath and presents a substantially smooth outer surface for use during dissection.

### BRIEF DESCRIPTION OF THE DRAWINGS

FIG. 1 is a side view of a prior art endoscope and dissector rod that are assembled axially.

FIGS. 2 and 3 are a cross-sectional representation of a dissector rod having rotatable parts allowing side entry of the endoscope into the dissector.

FIGS. 4 and 5 are a cross-sectional representation of an alternative embodiment having a hinged trap door allowing side entry of the endoscope into the dissector rod.

FIG. 6 is an end perspective view of a semi-cylindrical sheath member labeled to show how the semi-cylindrical shape can be quantified.

FIG. 7 is a front perspective view showing one preferred embodiment of an endoscope and dissector rod of the present invention prior to assembly.

FIG. 8 is a perspective view showing the endoscope and dissector rod of FIG. 7 after assembly.

FIG. 9 is a perspective view showing the assembled endoscope and dissector rod from the proximal end.

FIG. 10 is another perspective view showing the distal end in greater detail.

FIG. 11 is a cross-sectional view of the assembled endoscope and dissector rod.

FIG. 12 is a cross-sectional view showing the handle of the dissector rod in greater detail.

FIG. 13 is a side, perspective view of the dissector rod and tip including a seal for sealing the slit where the endoscope is radially inserted.

FIG. 14 is a cross section of the tip shown in FIG. 13.

FIG. 15 is a cross-sectional view showing an alternative embodiment having an additional seal receiving the endoscope at the distal end when closed.

FIGS. 16-18 are cross-sectional views of an alternative embodiment wherein the sheath comprises a resilient body that receives the endoscope off center.

FIGS. 19 and 20 are cross-sectional views showing a modification to the embodiment of FIGS. 16-18.

FIGS. 21 and 22 are partial cross-sectional views showing a duck-bill tip for use with the dissector sheath and endoscope of FIGS. 16-20.

### DETAILED DESCRIPTION OF PREFERRED EMBODIMENTS

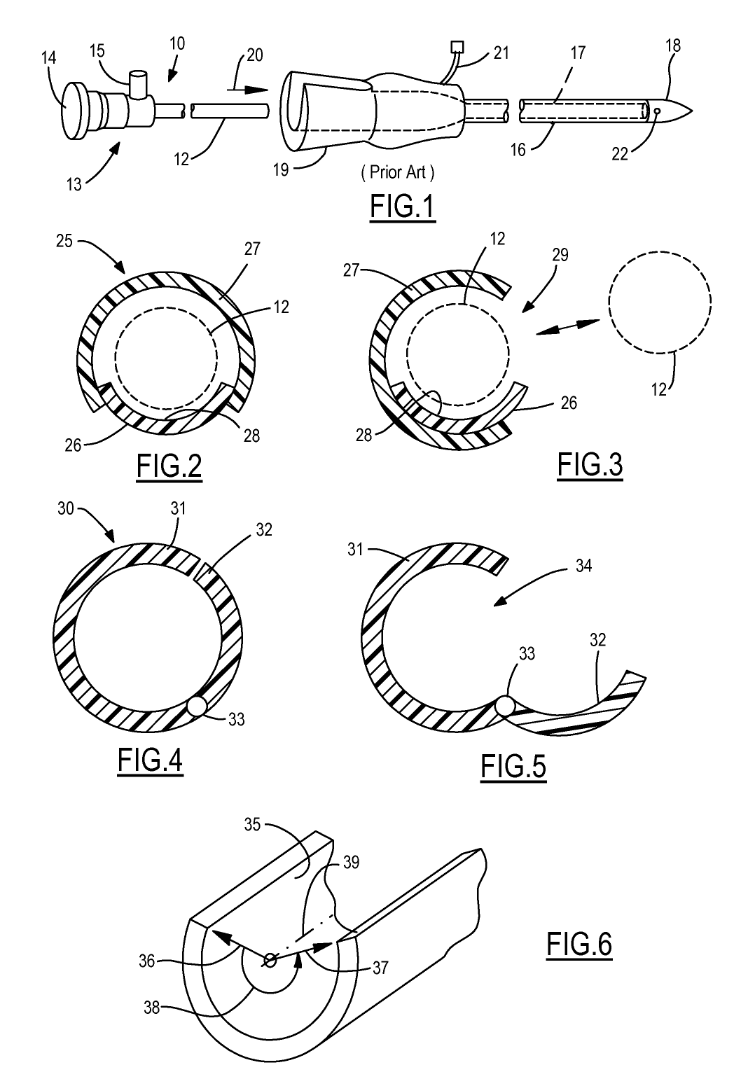

Referring now to FIG. 1, an endoscope 10 is shown together with a prior art dissector 11. Endoscope 10 includes a rigid endoscope rod 12 extending to a distal end and being connected to an endoscope head 13 at its proximal end. Head 13 includes an eyepiece 14 and a light connector 15. Dissector 11 includes a dissector rod having an outer sheath 16 with an internal bore 17. Sheath 16 is connected to a blunt dissector tip 18 at the distal end. A handle 19 at the proximal end of dissector 11 axially receives endoscope 10 (in the direction shown by arrow 20) so that endoscope rod 12 is inserted into bore 17 until it enters hollow tip 18 to allow visualization by the endoscope of an area being dissected through transparent tip 18. A space within bore 18 not occupied by endoscope rod 12 and/or additional passages within sheath 16 couple a gas inlet tube 21 at handle 19 with a gas outlet hole 22 at the distal end in either sheath 16 or tip 18 for the purpose of introducing insufflation gas to inflate a subcutaneous space around the dissection site. As previously explained, the axial insertion of the endoscope into the dissector has characteristics that may be undesirable in some situations.

The present invention solves the foregoing problems by providing a side-entry dissector. The dissector includes a generally-cylindrical sheath for retaining an elongated endoscope wherein the sheath is elongated between a distal end and a proximal end. A transparent tip is mounted at the distal end of the sheath. A handle is mounted at the proximal end of the sheath. The sheath includes a selectably closeable longitudinal slot having an open configuration such that the endoscope can be inserted radially into the sheath and has a closed configuration that retains the endoscope within the sheath and presents a substantially smooth outer surface for use during dissection.

A first embodiment shown in FIGS. 2 and 3 includes a dissector rod 25 show in cross section. FIG. 2 shows the closed configuration wherein a first sheath member 26 has a semi-cylindrical shape and creates a longitudinal niche to receive endoscope rod 12. First sheath member 26 is mutually rotatable around a longitudinal centerline with a second sheath member 27, which is also semi-cylindrical. FIG. 3 shows the sheath rotated into an open configuration which opens a longitudinal slot 29 which allows endoscope 12 to be radially inserted into longitudinal niche 28. After insertion of endoscope 12, the sheaths are rotated back into the closed configuration in order to present a smooth outer surface during use for dissection. After use, the sheaths are rotated to reopen longitudinal niche 28 through longitudinal slot 29 so that endoscope 12 can be removed for reuse and the dissector can then be disposed of.

A longitudinal slot for insertion of the endoscope can be created in other ways. For example, FIGS. 4 and 5 show an embodiment of a dissector rod 30 wherein a first semi-cylindrical sheath member 31 is connected to a second semi-cylindrical sheath number 32 by a hinge 33. When closed, the sheath members provide a continuous diameter with a substantially smooth outer surface for dissection. A locking mechanism (not shown) retains sheath member 32 in the closed configuration. The locking mechanism can be unlocked to open a longitudinal slot 34 as shown in FIG. 5.

A semi-cylindrical sheath member 35 is shown in FIG. 6 having a centerline 39. The shape of a semi-cylindrical member is characterized by a cylinder angle 38 between a radial line 36 from centerline 39 to one edge of the cylinder opening and a radial line 37 from centerline 39 to the other edge of the cylindrical opening. For purposes of the embodiment of FIGS. 2 and 3 with mutually rotating sheaths, the cylinder angles of the first and second sheath members must add up to greater than 360° in order to be able to create a closed cylinder. When the first sheath member directly provides a longitudinal niche on its inner surface on which the endoscope rod rests, then the cylinder angle for the first sheath member is typically less than or equal to about 180° so that the endoscope rod can be inserted into the niche. The cylinder angle of the second sheath member which is radially outward from the first sheath member is typically greater than about 210° in order to ensure a sufficient overlap of the sheath members when in the closed configuration. Most preferably, the second cylinder angle of the second sheath member is greater than or equal to about 270° but is only as large as will retain a sufficient width of the longitudinal slot for receiving the endoscope rod.

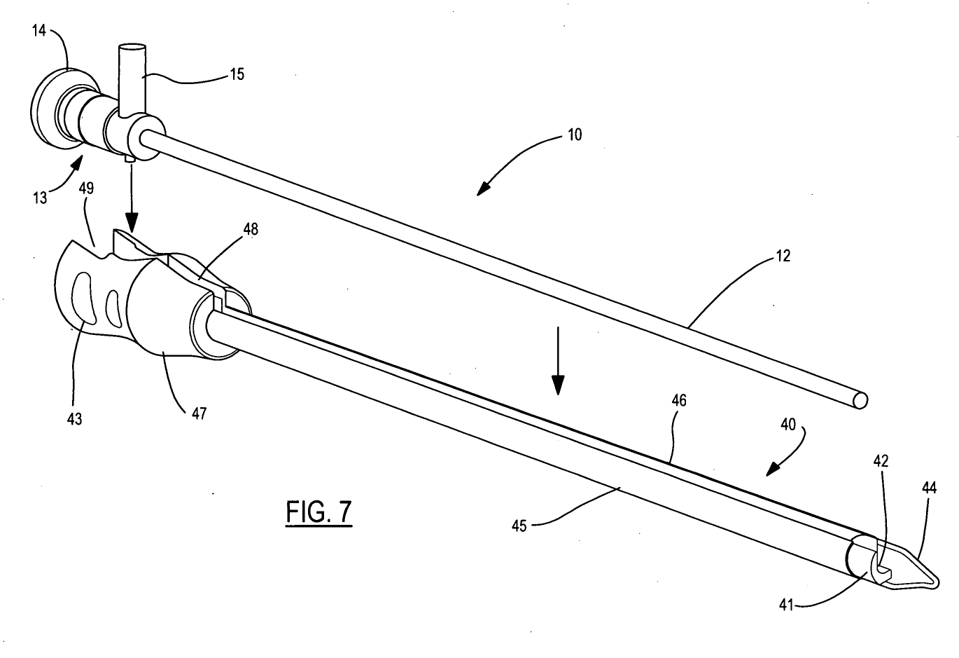

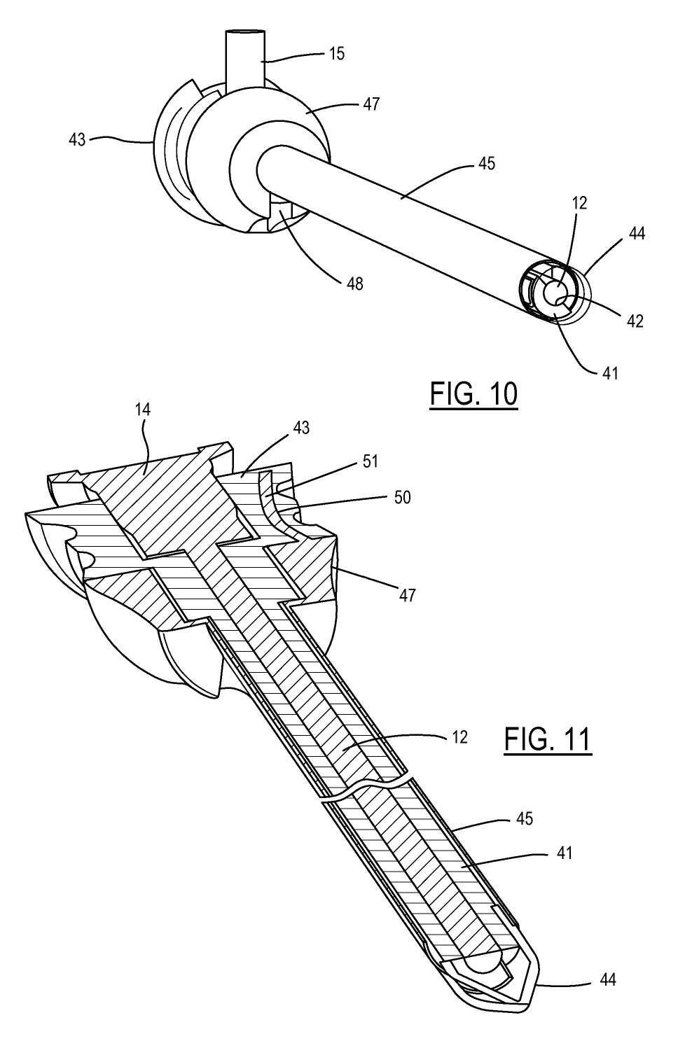

A more detailed embodiment and its operation are shown in FIGS. 7-14. A blunt dissector 40 is shown in FIG. 7 in its open configuration ready to receive endoscope 10 via a radial insertion. A first sheath member 41 has a niche 42 for receiving endoscope rod 12. Sheath member 41 is fixed with respect to a main handle 43 at its proximal end. A transparent tip 44 is provided at the distal end. A second sheath member 45 is disposed over most of the length of first sheath 41 and provides a longitudinal slot 46 through which endoscope 10 can be inserted into niche 42. Second sheath member 45 is coupled to a handle extension 47 that is juxtaposed with main handle 43. Handle extension 47 may be integrally formed with sheath member 45 (e.g., they may be an integrally molded plastic part). Handle extension 47 has a gap 48 for receiving a corresponding portion of endoscope rod 12. Handle 43 includes a recess 49 for receiving head portion 13 of endoscope 10, and may include a snap locking feature (not shown) as known in the art.

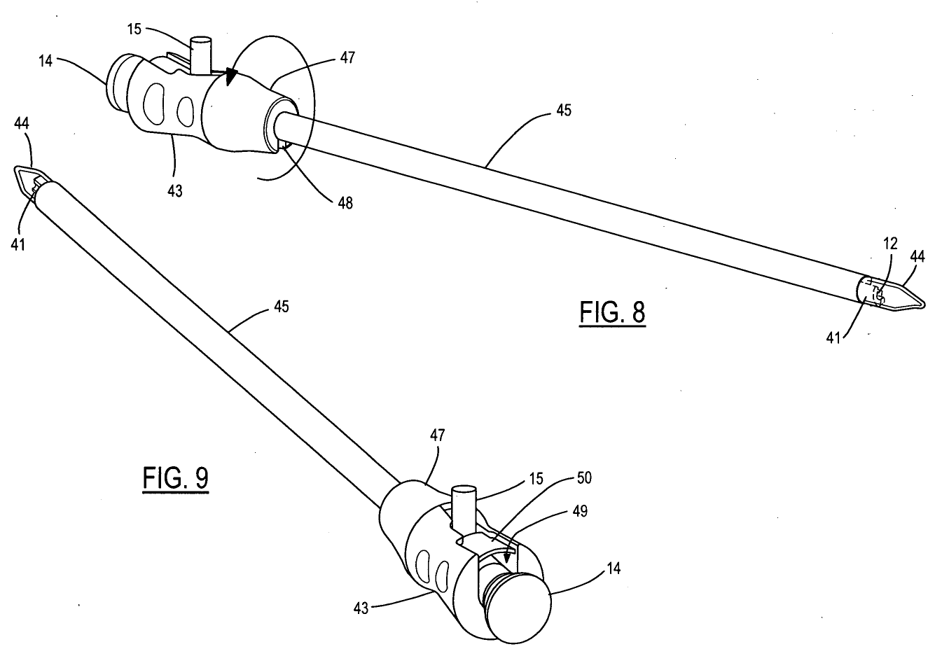

Handle extension 47 and second sheath member 45 mutually rotate with respect to first sheath 41 to enclose the endoscope within the dissector rod as shown in FIG. 8. Thus, gap 48 in handle extension 47 has been turned downward so that slot 46 in second sheath member 45 is shifted to the outer surface of first sheath member 41, thereby closing off the endoscope from the dissection environment and presenting a substantially smooth outer surface.

FIG. 9 shows the proximal end of the blunt dissector revealing a wing 50 formed as part of the handle extension which rotates to bridge recess 49 in order to assist in retaining the endoscope. Handle 43 includes an internal slot or groove (FIG. 11) for accommodating the wing so that wing 50 can be selectably positioned over the recess by rotating handle extension 47.

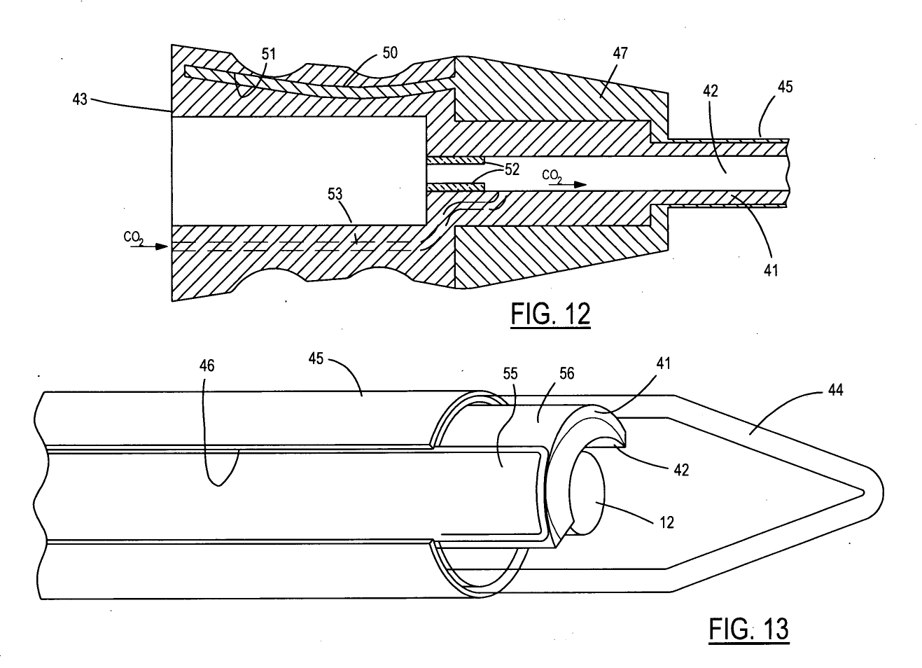

FIG. 11 shows a cross-section of the blunt dissector wherein wing 50 is seen extending from a main body portion of handle extension 47 into a corresponding internal slot 51 in handle 43. As shown in greater detail in FIG. 12, wing 50 may have a gradual arc shape. Handle extension 47 and wing 50 may preferably be comprised of a flexible material that allows handle 43 and handle extension 47 to be assembled by longitudinally inserting wing 50 into slot 51.

The present invention preferably includes fluidic seals so that the dissector is is waterproof along the longitudinal slot and/or at the tip to prevent entry of bodily fluids that could interfere with functioning of the endoscope. As shown in FIG. 12, an endoscope seal 52 is placed for receiving rod 12 to provide an airtight seal. A gas supply passage 53 can be coupled to an external supply of insufflation gas (not shown) such as CO₂. When rod 12 is inserted, the space between rod 12 and sheath member 41 provides a lumen for passage of the insufflation gas from to the distal end of the dissector.

FIG. 13 illustrates a resilient seal 55 that is bonded to an outer surface 56 of first sheath member 41. Resilient seal 55 may preferably be comprised of any biocompatible and sterilizable polymer or synthetic rubber, for example. Seal 55 has a longitudinal body extending along first sheath member 41 in order to extend through gap 46 of second sheath member 45 when the dissector is in its closed configuration. The thickness of seal 55 over outer surface 56 of first sheath member 41 is adapted to provide appropriate deformation against slot 46 for sealing, while permitting reopening of slot 46 by rotating sheath member 45 over seal 55.

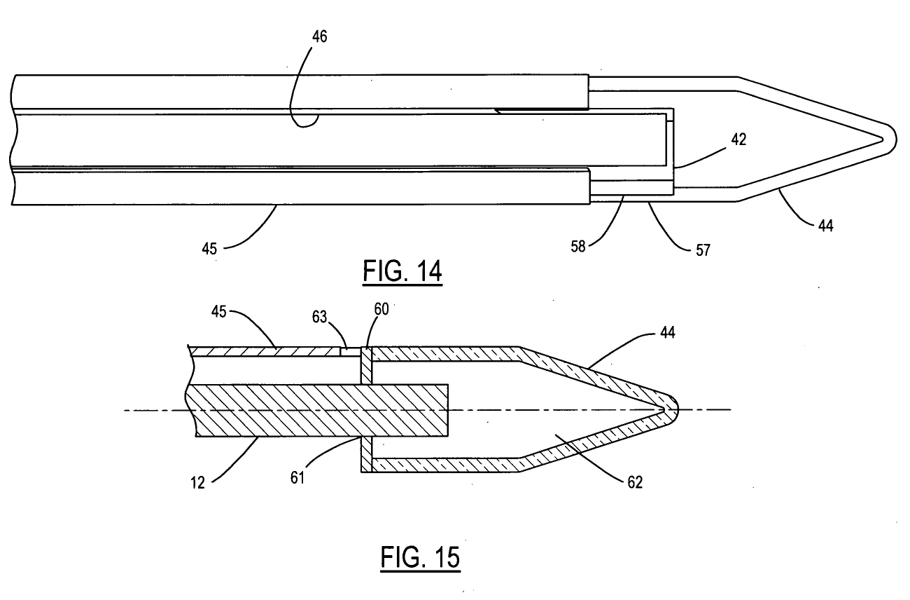

Dissector tip 44 is preferably attached to sheath member 45 as shown in FIGS. 13 and 14. Tip member 44 has a ledge 57 bonded to a tab 58 extending from second sheath member 45. Thus, tip 44 rotates with respect to first sheath member 42 along with sheath member 45. Longitudinal slot 46 does not extend the full length of first sheath member 42. Thus, a portion of niche 42 receiving the distal end of endoscope rod 12 extends within tip 44. Therefore, after radial insertion of the endoscope, it may be desirable to move it slightly in the axial direction to complete its installation into a position for obtaining a satisfactory view through tip 44.

As shown in FIG. 15, an annular seal 60 may be provided for preventing bodily fluids from entering tip 44 where they could interfere with the view through the endoscope. Seal 60 abuts tip 40 and/or sheath member 45 and has an inner opening 61 aligned with the centerline of the dissector. The inner diameter of opening 61 is less than the outer diameter of endoscope rod 12 so that there is an interference fit. Inner opening 61 receives the distal end of endoscope rod 12 so that a cavity 62 within tip 44 44 receives the end of endoscope rod 12 where it is sealed against intrusion of fluids during dissection. Seal 60 may be bonded between tip 44 and sheath member 45 as shown, or may be bonded to the inner annular surface of tip 44, for example. Sheath member 45 may also include a hole 63 that allows insufflation gas from within sheath member 45 to pass into the body cavity being dissected.

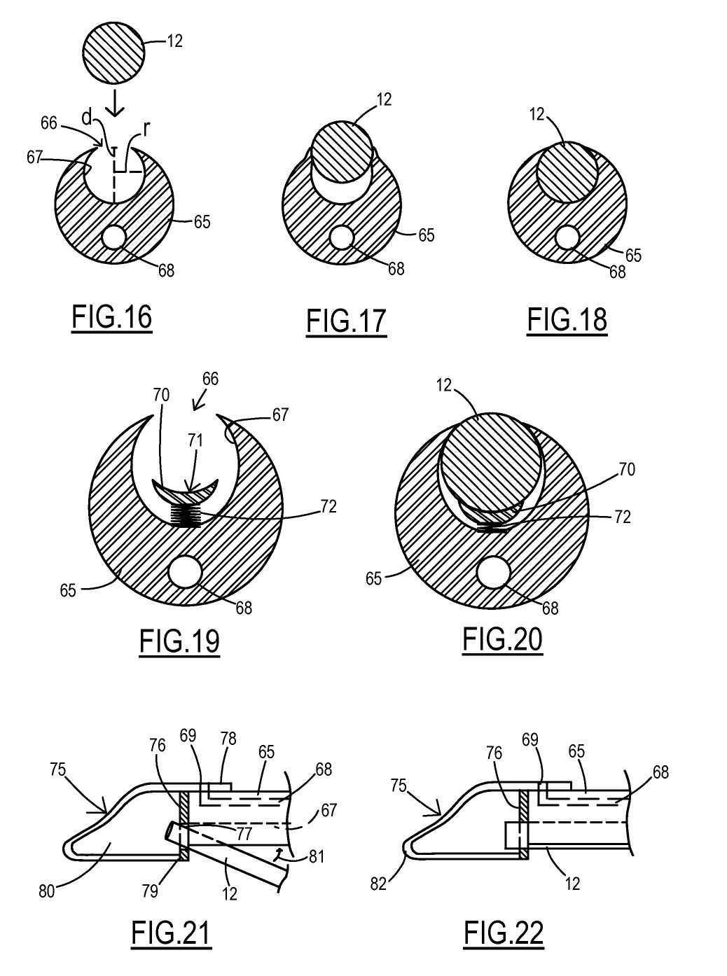

In an alternative embodiment shown in FIGS. 16-18, the sheath comprises a substantially solid body 65 formed of a resilient material. Preferably, the resilient material may be a fluoropolymer, such as polytetrafluoroethylene (PTFE, commonly known as Teflon), perfluoroalkoxy (PFA), fluorinated ethylene propylene (FEP), polyvinylidene fluoride (PVDF), ethylene-tetrafluoroethylene (ETFE), ethylene-chlorotrifluoroethylene (ECTFE), mixtures of fluoropolymers such as MFA or THV, or mixtures of any of the foregoing. The most preferred material for solid body 65 is PTFE. The use of a fluoropolymer reduces the friction caused by moving rod 65 through connective tissue, thereby reducing the force required to perform a dissection.

Sheath body 65 has an outer surface that is substantially cylindrical, and it has a longitudinal slot 66 which results from the creation of a substantially cylindrical bore 67 within body 65. Cylindrical bore 67 has a radius r and has a depth d into body 65 (i.e., bore 67 is off center so that it has an outer radial edge that protrudes through the outer substantially cylindrical surface of sheath body 65). The protrusion creates slot 66 such that the width of the slot is less than the diameter of endoscope 12. Since body 65 is made of a resilient material, it can be deformed along slot 66 in order to insert endoscope 12 in the radial direction as shown in FIG. 17. After insertion, body 65 assumes its original shape to securely retain endoscope 12. Furthermore, a substantially smooth outer surface is obtained because of the curvatures of body 65 and endoscope 12 and because endoscope 12 does not extend very far through slot 66.

Sheath body 65 also includes a longitudinal passage 68 for conducting insufflation gas between the proximal and distal ends. The proximal end of passage 68 communicates with a gas supply (not shown). At its distal end, passage 68 communicates with a hole 69 (FIGS. 21 and 22) in the tip that injects the gas into the body cavity being dissected, as known in the art.

FIGS. 19 and 20 show a modification that can be used to retain the endoscope more firmly and steadily within the bore. It uses a longitudinal receiver 70 mounted in substantially cylindrical bore 67. Receiver 70 has a crescent-moon shape creating a niche 71 for receiving endoscope 12. A continuous spring or a plurality of spaced springs such as a spring 72 are mounted to and between bore 67 and receiver 70. As shown in FIG. 20, spring 72 becomes compressed when endoscope 12 is received in niche 71. As a result, endoscope 12 is more stably supported within bore 67.

With endoscope 12 being offset from the center of sheath body 65 in the embodiments of FIGS. 16-20, the dissector tip must be modified accordingly. Preferably, a duck-bill shaped tip 75 is employed as shown in FIGS. 21 and 22. Tip 75 is transparent and is mounted to (e.g., bonded to) a sealing member 76 which is disk-shaped and has an off-center hole 77 for receiving the distal end of endoscope 12. Sealing member 76 is preferably bonded to the distal end of sheath body 65, an inside surface of a collar portion 78 of tip 75, and an edge surface 79 of tip 75. Tip 75 has an interior space 80 for receiving the distal end of endoscope 12 through hole 77. After placing the distal end of endoscope 12 into hole 77, endoscope 12 is radially inserted into bore 67 as shown by arrow 81 in FIG. 21. As seen in FIG. 22, the final position of endoscope 12 is in alignment with a distal end 82 of tip 75.

What is claimed is:

1. A blunt dissector for endoscopic vein harvesting comprising:
a generally-cylindrical sheath for retaining an elongated endoscope, wherein the sheath is elongated between a distal end and a proximal end;
a transparent tip at the distal end of the sheath; and
a handle at the proximal end of the sheath;
wherein the sheath includes a selectably closable longitudinal slot having an opened configuration such that the endoscope can be inserted radially into the sheath, and wherein the longitudinal slot has a closed configuration that retains the endoscope within the sheath and presents a substantially smooth outer surface for use during dissection.

2. The dissector of claim 1 wherein the handle includes a radial gap coinciding with the longitudinal slot for receiving a corresponding portion of the endoscope.

3. The dissector of claim 1 further comprising a fluidic seal associated with the longitudinal slot.

4. The dissector of claim 1 further comprising a fluidic seal at the distal end of the sheath for receiving a distal end of the endoscope and preventing fluids from entering the transparent tip during dissection.

5. The dissector of claim 1 wherein the sheath includes a substantially solid body formed of a resilient material, wherein the sheath has an outer substantially cylindrical surface, wherein the longitudinal slot is comprised of a substantially cylindrical bore within the substantially solid body, and wherein the substantially cylindrical bore has an outer radial edge protruding through the outer substantially cylindrical surface of the sheath.

6. The dissector of claim 5 further comprising:
a longitudinal receiver mounted in the substantially cylindrical bore and having a niche for receiving the elongated endoscope; and
at least one spring between the longitudinal receiver and the substantially solid body being compressed when the elongated endoscope is received in the niche.

7. A blunt dissector for endoscopic vein harvesting adapted to receive an endoscope having a head portion mounted at a proximal end of a rod portion, the dissector comprising:
a hollow handle having a recess for receiving the head portion;
a first sheath member fixed with respect to the handle and having a longitudinal niche extending around a longitudinal centerline from the handle toward a distal end of the first sheath member, wherein the longitudinal niche has an angular opening sufficient to accept radial insertion of the rod portion of the endoscope onto the centerline, wherein the radial insertion is substantially perpendicular to the longitudinal centerline;
a dissector tip mounted at the distal end of the first sheath member; and
a second sheath member movably mounted with respect to the first sheath member for enclosing the longitudinal niche after radial insertion of the rod portion into the niche for performing a dissection, and for reopening the longitudinal niche after performing the dissection to radially remove the rod portion from the first sheath member.

8. The dissector of claim 7 further comprising:
a handle extension coupled to the second sheath member and juxtaposed with the handle;
wherein the handle extension and the second sheath member are mutually rotatable around the centerline, and wherein the handle extension includes an external gripping surface for manually rotating the handle extension for selectably positioning the second sheath member over the longitudinal niche.

9. The dissector of claim 8 wherein the handle extension includes a wing, wherein the handle includes an internal slot receiving the wing, and wherein the wing is selectably positionable over at least a portion of the recess in the handle by rotating the handle extension.

10. The dissector of claim 7 wherein the first sheath member has a semi-cylindrical shape with an inner surface providing the longitudinal niche, and wherein the second sheath member has a semi-cylindrical shape with an inner surface nesting over an outer surface of the first sheath member.

11. The dissector of claim 10 wherein the first sheath member has a first cylinder angle, wherein the second sheath member has a second cylinder angle, and wherein the sum of the first and second cylinder angles is greater than 360°.

12. The dissector of claim 11 wherein the first sheath member has a first cylinder angle less than or equal to about 180°.

13. The dissector of claim 11 wherein the second cylinder angle is greater than or equal to about 210°.

14. The dissector of claim 11 wherein the second cylinder angle is greater than or equal to about 270°.

15. The dissector of claim 10 further comprising a resilient seal having a longitudinal body mounted to an outer surface of the first sheath member to extend through the second sheath member when it is enclosing the longitudinal niche.

16. The dissector of claim 7 further comprising:
a resilient annular seal abutting at least one of the dissector tip or the second sheath member and having an inner opening aligned with the centerline, wherein the inner opening is adapted to receive a distal end of the rod portion of the endoscope, whereby a cavity within the dissector tip containing the distal end of the rod portion during dissection is sealed against intrusion of fluids.
